(19) United States
(12) Patent Application Publication
(10) Pub. No.: US 2009/0279708 A1
(43) Pub. Date: Nov. 12, 2009

(54) ELECTRONIC STETHOSCOPE APPARATUS

(76) Inventor: Joseph Habboushe, Gladwyne, PA (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 12/306,161

(22) PCT Filed: Jun. 21, 2007
(86) PCT No.: PCT/US07/14487

§ 371 Date: Dec. 22, 2008

Related U.S. Application Data
(60) Provisional application No. 60/805,398, filed on Jun. 21, 2006.

(51) Int. Cl. A61B 7/04

(52) U.S. Cl. 381/67; 181/131

(57) ABSTRACT

An electronic stethoscope system that reduces cross-infection when examining a patient is provided. The electronic stethoscope system includes a handheld auscultation portion for picking up bodily sounds of a patient. The detected bodily sounds are transmitted to a base unit having a loudspeaker for broadcasting an audible representation of the detected bodily sounds. The sounds are transmitted to the base unit either by a wireless transmitter and receiver or by way of a disposable flexible tubing adapted for transmitting acoustic waves.

### I. FIELD OF THE INVENTION

The present invention relates generally to medical stethoscopes. More specifically, the present invention relates to an electronic stethoscope system and method for using the same to reduce cross-infection in hospitals.

### II. BACKGROUND OF THE DISCLOSURE

Hospital-acquired infections every year cause up to 90,000 deaths, 2 million extended hospital stays, and over $2.6 Billion in medical costs, in the U.S. alone. Research has been found that some up to 90% of conventional stethoscopes in a hospital carry infectious bacteria. This is especially significant for patients in isolation beds. Currently, hospitals put a cheap disposable stethoscope bedside as a means of limiting patient-to-patient transmission of infections.

However, a fully gowned and gloved physician must still break the isolation barrier to use these disposable stethoscopes. As multiple members of the physician team separately use the disposable stethoscope during the course of a patient's stay, this isolation barrier is broken several times. In addition, physicians often avoid using this cheap stethoscope bedside because of either poor sound quality and/or because of the discomfort to putting a stethoscope many other people may have used in their ears. Instead, these doctors either use their own stethoscopes, breaking the isolation barrier, or perhaps worse, they avoid routine stethoscope examinations.

Accordingly, there is a need for a stethoscope apparatus that reduces hospital-acquired infections and that physicians are readily disposed to use.

### III. SUMMARY OF THE DISCLOSURE

Instead of the one-stethoscope-per-doctor standard, the present invention is directed to one-stethoscope-per-patient. The earpieces are done away with—instead, the diaphragm is connected to tubing which goes straight to a base unit, which includes a speaker and a display monitor. Sound is played over the speaker and displayed graphically, with ability to save the electronic information, upload it to medical records, as well as immediately analyze the sound with diagnosis software, much like an EKG machine. The diaphragm and tubing may be manufactured as disposable pieces, changed for every new patient, or even for every new examination of the same patient. Alternatively, the diaphragm an tubing may be manufactured in a manner allowing them to be sterilized between patients or examinations.

While the stethoscope examination is not a gold standard medical test, it is a common screening exam used often throughout a hospital because it is quick and easy. Therefore, in order for the present invention to be valuable and successful device, it must be VERY easy to use, with a great user interface, in reach of the examining physician, and easily adaptable in the hospital and clinic.

The electronic stethoscope of the present invention hooks up directly to a speaker and display system, and hangs bedside. In such a way, the physician need not break isolation to use the stethoscope; he or she can both hear and see the sounds on the wall unit; all healthcare providers on the team can listen and see the results at the same time, so the stethoscope need only be used once per visit. There is no reason for the physician to use his or her own stethoscope, and no reason to avoid this step of the exam. The disposable tubing, and possibly the diaphragm piece, of the present invention is changed for every patient, so one method of hospital-acquired infection is eliminated.

The sounds data is played over a speaker, as well as displayed on a monitor bedside. Software may be provided in the base unit for analyzing the bodily sounds and make diagnosis, much like an EKG machine. The processed sounds, in the form of electronic signals, can be easily recorded, and uploaded to electronic medical records for later review and archiving. The many benefits of these include improving the quality of healthcare, reducing the cost, saving time, and even improving teaching.

Additional benefits that may be realized by the present invention include: a reduction in ambiguity regarding whether a current exam result is better or worse than a previous one, as reliance on a different doctor's analysis is no longer necessary—a patient's current physician can just refer back to the stored audio or visual file, or software report. Telemedicine is another benefit of the present invention, for example, a nurse could perform the exam, and a doctor would then be able to review the audio later from either his or her office or at some other remote location.

Moreover, use of expensive, time-consuming tests that often have deleterious side effects can be reduced or even eliminated. For example, there may be specific borderline situations in which a physician currently would send for an ECHO or CT Scan “just to be sure,” which he may not feel the need to do when the electronic stethoscope of the present invention makes the stethoscope exam more reliable. This will result in a reduction in time, cost, and side effects (such as radiation from CT).

### IV. BRIEF DESCRIPTION OF THE DRAWINGS

These and other features, aspects, and advantages of the present invention will become better understood with regard to the following description, appended claims, and accompanying drawings wherein:

FIG. 1 illustrates a representation of an embodiment of the present invention;

FIG. 2 illustrates a cross-sectional view of a disposable tubing for coupling the auscultation portion with the base unit of the embodiment of the present invention shown in FIG. 1;

FIG. 3 illustrates a block representation of a wireless embodiment of the present invention; and

FIG. 4 illustrates a flow diagram for using an embodiment of the present invention to reduce cross-infection during stethoscopic examinations.

### V. DETAILED DESCRIPTION OF DISCLOSURE

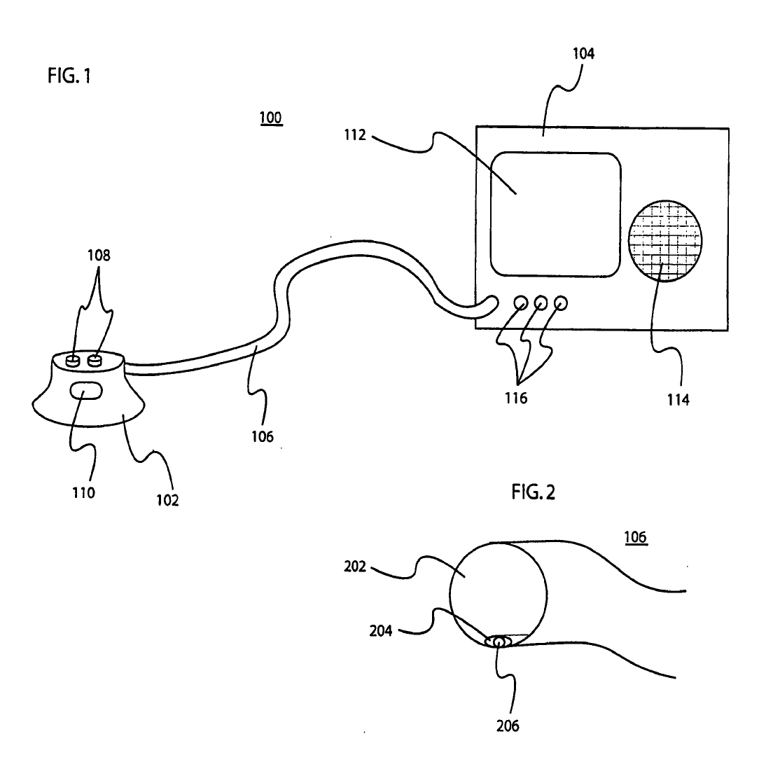

Referring to FIG. 1, an electronic stethoscope system 100 is shown. The electronic stethoscope system 100 has a handheld auscultation portion 102 in audio communication with a base unit 104. A disposable flexible tubing 106 couples the auscultation portion 102 to the base unit 104.

The auscultation portion 102 is dimensioned to comfortably fit in a physician hand. Several controls 108 may be disposed on a top surface of the auscultation portion 102 positioned with easy access of the controls 108 to a physician's fingers. Additionally, thumb controls 110 may be situated on a side of the Auscultation portion for actuation by a physician's thumb. The auscultation portion 102 is equipped with a sound pick-up device disposed to pick up bodily noises from a patient. Sound pick-up devices that are envisioned as applicable in the present invention include thin diaphragms, microphone devices, or other devices capable of picking up a patient's bodily sounds.

In the case of the microphone device, or other electronics based sound pick-up device, the disposable tubing 106 is replaced with either a disposable cable containing a plurality of electrical wires, or a cable capable of being sterilized in a manner commonly known in the art.

Alternatively, If a diaphragm is used, the sounds received by the diaphragm can be converted to digital representations within the auscultation portion 102 and transmitted by way of a cable containing a plurality of wires, as described above, The wires transmit the digital sound data to the base unit 104.

The auscultation portion can be provided in multiple shapes and sizes depending on the particular use, such as pediatric versions, veterinarian versions, etc, or adapted for providing Doppler functionality to allow auscultation of arterial blood flow or fetal heart beat. An ultrasound version is also envisioned, which includes an ultrasound transducer for emitting and receiving ultrasound signals. Moreover, the auscultation portion may come equipped with a magnet for detecting metal within a patient's body, such as foreign objects and medical devices—for example, ICDs, pacemakers, indewelling catheters, stents, feeding tubes, intubation tubes, nasal gastric tubes, etc.

The disposable tubing 106 is adapted for transmitting audio waves from the auscultation portion 102 to the base unit 104. The disposable tubing allows a hospital worker to replace the disposable tubing for each new patient so that a new piece of tubing 106 is used for each patient. Further, the tubing 106 can be replaced between each examination, reducing the chances of cross infection even more.

The base unit 104 has a loudspeaker 114 for broadcasting audio corresponding to bodily sounds picked up by the auscultation portion 102. The loudspeaker allows multiple physicians to listen to the bodily sounds picked up by the auscultation portion simultaneously. Additionally, the base unit 104 includes a display 112, which displays diagnostic representations of the bodily sounds, allowing physicians to visually evaluate the sounds.

The base unit 104 contains an acoustic signal processing circuit for converting the audio waves received by the auscultation portion into electronic representations, which can then be further processed for display on the display 112 and evaluated by diagnostic software capable of making diagnoses suggestions, for example the system would be able to judge a heart sound as a III/VI mitral valve murmur.

Further, the base unit 104 may be equipped with a user interface (not shown), such as a touch screen overlay on the display 112, or keyboard and pointing device, which allows a healthcare worker to identify the patient being examined, which part of a patient's anatomy is being examined (e.g. heart, which part of heart, or lung, which quadrant of lung), time and date, other diagnostic devices being used, etc.

The controls 108 and 110 may also be configured to provide user inputs to the base-unit 104, and in fact, this option may be preferable as it eliminates the need for the physician to touch anything other than the auscultation portion 102. This information will be saved along with the sound and video files. These files are downloadable onto computer media, saved in speaker/display, or uploaded into medical records. The base unit 104 is further equipped with controls 116 for allowing a physician or healthcare worker to select different frequency and/or amplitude ranges for the sounds to be displayed.

The base unit 104 can be equipped with prompts requiring the healthcare worker to provide information regarding where each step of the exam is being done. For example, it may ask that the healthcare worker first put the diaphragm on the right sternal border, and then a few moments later move it to the left sternal border, etc. It can prompt for bell vs. diaphragm use. For lung examinations, the prompts may request different lung fields. In this way, a stethoscopic examination can be carried out in a very controlled and uniform manner, reducing the chance that a particular area of interest is not examined. The prompts may be presented either on the display 112 or by way of speech synthesis. In addition response may be provided using voice commands as well as through actuation of the controls 108, 110 and 116 on the auscultation portion 102 and base unit 104.

Also, the base unit 104 can be configured to store voice annotations from the examining healthcare worker along with the stethoscopic sounds. In this way, notes regarding the examination can remain associated with the recorded stethoscopic sounds. By saving such exams, future healthcare workers will have data to refer to as a baseline, to see if patient conditions have worsened or are stable.

The base unit 104 may be wall mounted, attachable to a patient's bed, built into the patient's bed, free-standing, incorporated with other bedside monitors, remotely located, portable, and mountable in an ambulance. The base unit 104 may further provide connectors for storage media such as SD cards, MM cards, flashdrives, etc. for downloading examination data and reports from the base unit 104. This interface may also be configured to allow upload of audio files as well as for updating firmware, etc.

Additionally, the base unit may be networkable, i.e., connectable to a hospital's local area network, wide area network or the Internet, allowing remote users to receive data from the base unit 104, as well as allowing the examining healthcare worker to retrieve information through the base unit 104, such as patients records stored at an in-hospital database server or from other hospitals that may have provided treatment to the patient.

The base unit 104 may come equipped with headphone jacks to allow a healthcare worker to plug in a pair of headphones in situations such as emergency rooms where the noise level may make it difficult to properly listen to the stethoscopic sounds broadcast by the loudspeaker, or in situations where there is a risk of disturbing nearby patients. Wireless headphones may also be usable with the base unit 104 by providing a wireless transmitter adapted for connecting to wireless headphones.

Moreover, the base unit 104 may be dimensioned with a dispenser section configured for holding a plurality of the disposable portions of the present invention, such as disposable tubing an/or auscultation portions, in their sterile packaging. This provides the healthcare worker with sterile components for the present invention conveniently located near the base unit 104.

Returning to the controls 106 and 110 disposed on the auscultation portion 102, these controls, may be configured as buttons, levers, dials, trackballs, or any other control type commonly known in the art. These controls can be adapted for controlling such properties as volume, power, pitch, and normal or bell diaphragm modes. Additionally, the controls may be configured to control recording, playback and speed of playback, mute, provide inputs to menus and prompts provided by the base unit 104, and may provided user customizable features as well. The present invention may come pre-configured with presets optimized for specific types of examination, e.g., heart, lung, abdominal, etc.

Further, indicators such as light emitting diodes and liquid crystal displays may be incorporated into the auscultation portion 102 for providing useful information to the examining healthcare worker without the need for the healthcare worker to turn away from the examination. Such information may include signal strength, to inform the examining healthcare worker if the sounds being received are strong enough for the system to analyze and provide a diagnosis.

Ideally, the various controls should be differentiable to the user by tactile senses alone, so that when the physician is using the auscultation portion to listen for lung sounds, for example, and thus has the auscultation portion held in a manner in which it is impossible to identify the controls by sight, the physician is still able to actuate the controls as desired.

Alternatively, the controls may be moved from the surface of the auscultation portion to a separate remote control unit actuated either by hand or foot and in communication with the base unit 104. The controls may also be duplicated on the front panel of the base unit 104 as well, either by providing physical controls or by way of the previously mention touch-screen interface.

Controls may also be provided that allow the examining healthcare worker to transmit the results of the examination, either sound and video files or full electronic medical records, to specified locations, such as a nurses station or doctor's office.

Essentially, the purpose of the controls provided by the present invention is to create an examination environment, which is easily adaptable to an individual healthcare worker's workflow, thus increasing productivity and usability of the electronic stethoscope.

Turning to FIG. 2, a cross-section of the disposable tubing 106 is shown. The disposable tubing 106 is constructed of a flexible material such as rubber, plastic, etc., such that a central cavity 202 is formed that runs the length of the tubing 106. The cavity 202 allows acoustic waves to propagate along the tubing 106 from the auscultation portion 102, coupled at one end, to the base unit 104, coupled at the opposite end.

Additionally, the tubing 106 has a second cavity 204 formed adjacent to a wall of the tubing 106. The secondary cavity 204, also running the length of the tubing 106, houses a plurality of wires 206 having electrical contacts (not shown) formed at both ends of the tubing 106. The wires 206 provide transmission of electrical signals between the auscultation portion 102 and the base unit 104. The electrical signals providing control and energizing power between the auscultation portion 102 and the base unit 104.

The wires 206 can either run within the rubber of the tubing 106, within the cavity 202 of the tubing 106, or outside the tubing 106 attached to the outside surface. The end of the tubing 106 that fits into the auscultation portion 102 and the base unit 104 provide contacts for these wires 206. This may be achieved by having a hard surface, such as a metal ring, at the end of the rubber tubing, which is continuous with the wires 206 running with the tubing 106 and also comes in contact with a similar surfaces on the auscultation portion 102 and base unit 104, allowing the electrical signals to be transmitted between the auscultation portion 102 and the base unit 104. Other techniques for providing electrical contact between the wires 206 and the auscultation portion 102 and the base unit 104 are well known in the art and suitable for the present invention.

In the embodiment of the present invention where the disposable tubing 106 is used, the standard auscultation head of conventional stethoscopes may be used in place of the above disclosed auscultation portion 102, as the sound is transmitted in the conventional manner within the disposable tubing 106 and converted to digital form at the base unit 104.

Alternatively, an attachment piece housing an analog-to-digital (A/D) converter and either wireless transmitter or connector cable for electrically connecting to the base unit may be removeably attached to a conventional auscultation head as well. In this case the attachment piece converts the analog stethoscopic sounds into digital representations by way of the A/D converter and transmit the digital signals to the base unit 104 by way of the wireless transmitter or cable. The attachment piece can be disposed with all the controls and indicators discussed above as being part of the auscultation portion 102, thus converting a conventional auscultation head into a full-featured auscultation portion of the present invention.

A further alternative is to provide a conventional electronic stethoscope with wireless transmission capabilities at the time of manufacture allowing the so equipped conventional electronic stethoscope to communicate with the base unit 104 of the present invention. This allows a physician with a so equipped personal conventional electronic stethoscope to use his stethoscope in conjunction with the base unit 104 of the present invention.

Preferably, the base unit 104 is capable of accepting both tubing based and electric cable based auscultation portions. This is possible using the same coupling interface described above simply by providing additional contacts on the base unit 104 side to provide connections for wires carrying digital sound data as well as the control and power contacts. Thus if signals are received on the sound data contacts, the base unit 104 will operate in a wired mode, while in cases where no signal is received on the sound data contacts, the base unit 104 will operate in an analog acoustic wave input mode, i.e., tubing mode. Moreover, if the base unit 104 includes a wireless transmitter than receipt of data over the wireless transmitter will result in the base unit 104 operating in a wireless mode. These modes can be selectable by the user by way of menu options or physical control settings, as well.

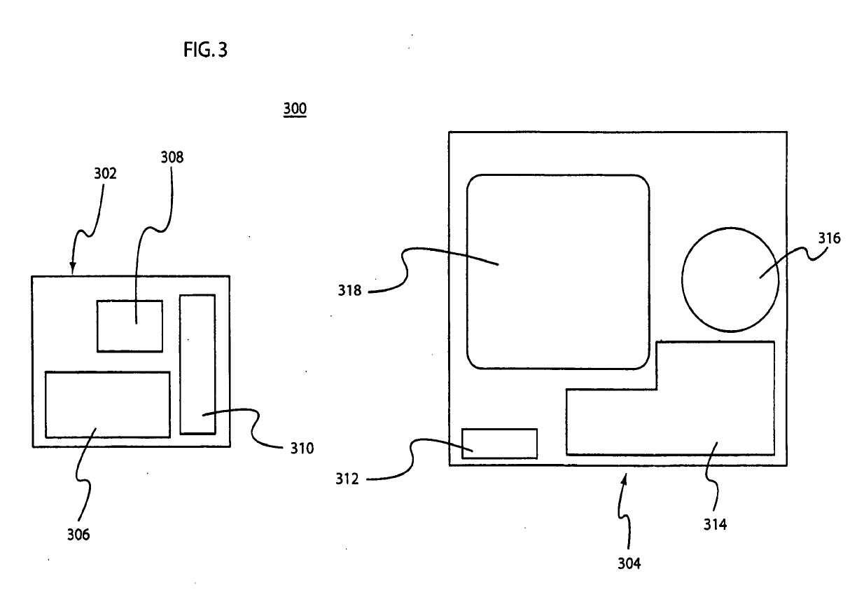

Referring to FIG. 3, a block representation is shown of an alternative implementation of the present invention. Specifically in this implementation, the tubing 106 of FIG. 1 is replaced with wireless transmitting and receiving units 310 and 312 in the auscultation portion and base unit, respectively.

The wireless version of the electronic stethoscope 300 of the present invention is equipped much like the embodiment shown in FIG. 1, having an auscultation portion 302 and a base unit 304. The auscultation portion 302 contains a sound pickup device 306, such as a diaphragm or microphone, coupled to an analog-digital converter circuit 308. The digital representations of the picked-up sounds are broadcast to the base unit 304 using a wireless transmitter 310 in the auscultation portion. In addition, the transmitter 310 is also adapted for transmitting and receiving control signals between the auscultation portion 302 and the base unit 304.

The base unit 304 includes a wireless transmitter 312 adapted for transmitting and receiving wireless signals from the auscultation portion 302. Received wireless signals are transmitted to a signal processor circuit 314, which determines whether the signal is a control signal or a digital acoustic signal and process the received signal accordingly. For example control signals may be used to respond to prompts issued by the base unit or to cycle through diagnostic options, etc., while the digital acoustic signals are further processed for broadcast through the loudspeaker 316 and display on the display 318.

Additionally, the signal processing circuit 314 may include diagnostic function for identifying specific pathological sounds and abnormalities. The base unit 304 may present the tentative diagnosis on the display 318 along with a visual representation of the acoustic signal. The signal processing circuit 314 can process the audio signal to remove or reduce background noise, thus making the recognition of specific pathological sounds and abnormalities easier.

Additional monitoring functions can be provided in the base unit 304, such as monitoring the sounds for any changes, and report detection of any changes or detected pathologies by way of audible beeps, voice synthesis, on-screen messages on the display 318. The base unit 304 can also notify the hospital nurses station or other designated remote location of the changes. Moreover, the base unit 304 may be configured to begin administering therapeutic treatments or additional diagnostics based on the detected changes. In this case, the base unit 304 is adapted to control additional medical equipment in proximity to the base unit 304 and patient.

The wireless technology used for the transmission may be Bluetooth, Infrared, WiFi, RF, or any other suitable wireless technology having the requisite range and data throughput. Additionally, encryption technologies may be employed to reduce the risk of unauthorized persons intercepting the signals and compromising a patient's privacy.

Not shown in FIG. 3, but still considered part of the present embodiment are the controls 108, 110 and 116 disposed on the auscultation portion 302 and base unit 304 as shown in FIG. 1.

Furthermore, a version of the present invention having multiple auscultation portions is envisioned. The multi-auscultation version is disposed with a plurality of auscultation portions that can be affixed to a patient for continuous monitoring of bodily sounds. Such monitoring can be useful in determining the recovery of a post-operative patient. The auscultation portions may be positioned to listen for sounds at all abdominal quadrants.

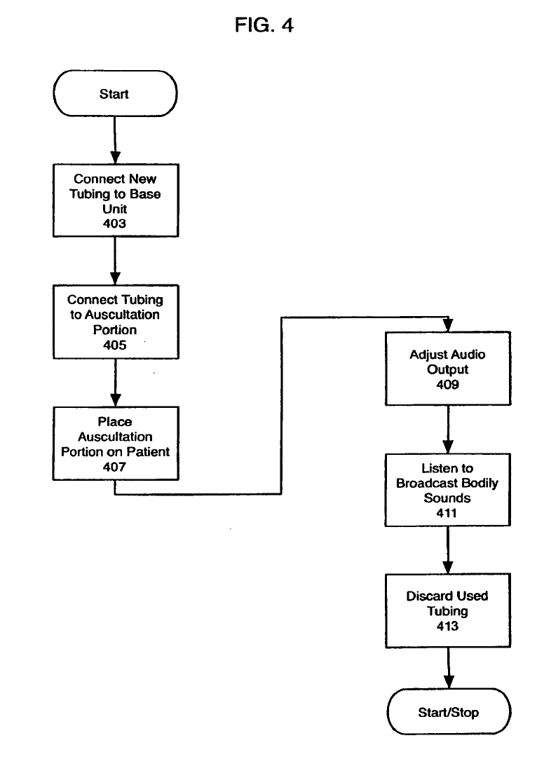

Referring to FIG. 4, a method is shown for using the electronic stethoscope of the present invention in a manner that reduces the risk of cross contamination to a patient during a stethoscopic examination. At the initiation of the examination, a healthcare worker retrieves a sterile stethoscopic tubing and couples the tubing to the base unit in step 403. The opposite end of the tubing is coupled to the auscultation portion in step 405. As part of steps 403 and 405, the control wires embedded in the tubing are connected to corresponding contact points on the base unit and auscultation portion.

The healthcare worker places the auscultation portion on a target area of a patient's anatomy for picking up bodily sounds in the target anatomical area in step 407. The healthcare worker adjusts the audio output, if necessary, of a loudspeaker housed in the base unit in step 409. Next the healthcare worker listens to the broadcast bodily sounds and views visual representations of the bodily sounds in step 411.

Steps 407 through 411 may be repeated a number of times at different target areas in the course of the examination. Upon completion of the examination, the healthcare worker disconnects the stethoscopic tubing from the auscultation portion and base unit, and discards the tubing in an appropriate manner in step 413. By following these steps a healthcare worker can greatly reduce the risk of infecting the patient.

Through the improvements to the conventional stethoscope provided by the present invention, increase in its use, and standardization of the physical diagnosis through software interpreters and ability to save data, the present invention may reduce the number of needed “further tests” such as echocardiograms and high-resolution CT scans, particularly in borderline cases. For example, if a patient were found to have a heart murmur in 2006, and given an echocardiogram. Three years later, a new physician examining the patient hears a heart murmur, and looking through the records sees that the patient had an echocardiogram. However, this physician may not be able to tell if the murmur has become worse—as he can only compare his own interpretation of the exam with that jotted down by the first doctor. However, had the first stethoscope exam by the first doctor been done with the electronic stethoscope of the present invention, the degree of murmur would have been standardized, and the sound and display files may even be available for review. A repeat echocardiogram may not be necessary.

Similar scenarios often occur with lung sounds and High Resolution CT scans, which are not only expensive and time intensive tests, but also expose the patient to a large amount of radiation—radiation which may be avoidable with the present invention.

On the other side of the coin, the stethoscope of the present invention will decrease the amount of missed diagnoses, as less trained healthcare workers are more likely to catch remarkable findings of the physical exam when stethoscope-received sounds are played over a speaker, displayed on a screen, and analyzed with software.

For the same reason, the present invention will be usable as a training aid for healthcare workers to hear the differences in stethoscope sounds, as the software will analyze and tell the healthcare worker what they are hearing.

Furthermore, as the sound is played over a speaker and displayed visually, it will be unnecessary for each member of a physician's team to listen with his or her own stethoscope, saving time bedside, as well as saving the patient the hassle of having each member of the team touch him or her.

Since the stethoscopic sounds are saved electronically as a sound file and a video file, the system of the present invention can prove extremely valuable in the increasing use of “telemedicine,” which allows physicians to review sounds of a patient before even meeting the patient, while now the physician may only be able to review medical records, medical images, etc.

Moreover, the telemedicine applications of the present invention may provide a significant upgrade in the quality of medical service available in remote places, such as mobile medics in the 3^{rd} world; space stations; polar stations; submarines; cruise ships.

The present invention may include pre-saved standard sounds for review, such as “normal” heartbeat, and specific pathologic sounds (lung sounds, abdominal sounds, etc). This feature may be incorporated into a “learning mode” as well, with exercises and demonstrations to increase a physician's or healthcare worker's ability to detect and distinguish specific pathological sounds. For example, normal active bowel sounds signal that the gut is starting to work again after having surgery. No bowel sounds or high-pitched sounds are consistent with a bowel obstruction. Being able to properly distinguish between these sounds may greatly impact a patient's recovery time.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

What is claimed is:

1. A electronic stethoscope system, said system comprising:
means for detecting bodily sounds housed in a handheld unit;
means for controlling said electronic stethoscope system;
means for transmitting said detected bodily sounds to a base unit; and
means for presenting said detected bodily sounds in a form perceivable to a physician proximate to said base unit, said presenting means being disposed on said base unit.

2. The system as in claim 1, wherein said transmitting means is selected from a group consisting of: a flexible tubing adapted for transmitting bodily sounds from said auscultation unit to said base unit as acoustic waves, a wireless transmitter, and a plurality of electrically conductive wires.

3. The system as in claim 2, wherein said flexible tubing includes at least one wire affixed thereto for providing power to said controlling means and control instructions between said controlling means and said base unit.

4. The system as in claim 1, wherein said controlling means includes a plurality of input means disposed on said handheld unit.

5. The system as in claim 1, wherein said controlling means includes a voice control means, for controlling said system based on spoken commands issued by said physician.

6. The system as in claim 1, wherein said controlling means includes a plurality of input means disposed on said base unit.

7. The system as in claim 1, wherein said controlling means includes user controls for adjusting sound properties for said presenting of said detected bodily sounds.

8. The system as in claim 1, further comprising a storage means for storing examination information, including patient identification, part of the anatomy examined, audio file and video file corresponding to said bodily sounds, and diagnosis information.

9. The system as in claim 1, further comprising a diagnosis means for evaluating said bodily sounds and presenting to said physician a possible diagnosis.

10. The system as in claim 1, wherein said presenting means is selected from a group consisting of a video display, loudspeaker, and a combination of both.

11. The system as in claim 10, wherein said video display includes a touch-sensitive screen in communication with said controlling means for accepting user commands by why of user selection of displayed options.

12. The system as in claim 1, further comprising a network connecting means for connecting to a network, said system being configured for transmitting and receiving patient data and medical related data over said network connection means.

13. The system as in claim 1, wherein said system is integrated into a hospital bed.

14. The system as in claim 1, further comprising one or more accessory handheld units for selectably replacing said handheld unit, said accessory handheld units selected from the group consisting of: said handheld units of differing dimensions, a Doppler head, an ultrasound head, and a plurality of handheld units.

15. An electronic stethoscope system, comprising:
an auscultation unit disposed in a handheld housing;
a base unit in communication with said auscultation unit;
a sound reproduction element disposed in said base unit, said sound reproduction device configured for reproducing sounds received from said auscultation unit into a form perceptible to a treating physician;
user controls disposed on a surface of said handheld housing for controlling at least properties of said auscultation unit and said sound reproduction element.

16. The system as in claim 15, wherein said sound reproduction element is selected from a group consisting of a video display, loudspeaker, and a combination of both.

17. The system as in claim 16, wherein said video display includes a touch-sensitive screen in communication with said user controls for accepting user commands by way of user selection of displayed options.

18. The system as in claim 1S, further comprises a transmitter and receiver for transmitting bodily sounds from said auscultation unit to said base unit, wherein said transmitter is selected from a group consisting of: a flexible tubing adapted for transmitting bodily sounds from said auscultation unit to said base unit as acoustic waves, a wireless transmitter, and a plurality of electrically conductive wires.

19. The system as in claim 18, wherein said flexible tubing includes at least one wire housed therein for providing power to said auscultation unit and control instructions between said auscultation unit and said base unit.

20. The system as in claim 15, wherein said controlling means includes user controls for adjusting sound properties for said reproduction of said detected bodily sounds.

21. The system as in claim 15, further comprising a storage section for storing examination information, including patient identification, part of the anatomy examined, audio file and video file corresponding to said bodily sounds, and diagnosis information.

22. The system as in claim 15, further comprising a diagnosis processor for evaluating said bodily sounds and presenting to said physician a possible diagnosis.

23. The system as in claim 15, further comprising a network connector for connecting to a network, said system being configured for transmitting and receiving patient data and medical related data over said network connector.

24. The system as in claim 15, wherein said system is integrated into a hospital bed.

25. The system as in claim 15, further comprising one or more accessory handheld units for selectably replacing said auscultation unit, said accessory handheld units selected from the group consisting of: said handheld units of differing dimensions, a Doppler head, an ultrasound head, and a plurality of handheld units.

26. The system as in claim 15, further comprising a voice control system for accepting voice commands from a physician and performing functions designated by said voice commands.

27. A method for reducing cross-infection when performing a stethoscopic examination, comprising the steps of:
retrieving a sterile stethoscope tubing at initiation of said stethoscopic examination;
coupling a first end of said stethoscope tubing to a handheld auscultation unit;
coupling a second end of said stethoscope tubing to a base unit;
placing auscultation unit on a target site of a patient's anatomy receiving bodily sounds with said auscultation unit;
listening to said bodily sounds broadcast by a speaker housed in said base unit; and
replacing said stethoscope tubing upon completion of said stethoscopic examination.

28. The method as in claim 27, further comprises the step of viewing a visual representation of said bodily sounds on a display housed in said base unit.

29. The method as in claim 27, further comprising the step of coupling a plurality of wires, affixed to said stethoscopic tubing, to said auscultation unit at said first end of said tubing and to said base unit at said second end of said stethoscopic tubing, said plurality of wires forming an electrical communication link between said auscultation unit and said base unit for transmitting control and energizing signals therebetween.

30. The method as in claim 29, wherein said control signals includes signals for controlling sound properties for said of said bodily sounds being broadcast.

31. The method as in claim 27, further comprising the step of storing examination information, including patient identification, part of the anatomy examined, audio file and video file corresponding to said bodily sounds, and diagnosis information.

32. The method as in claim 27, further comprising the step of evaluating said bodily sounds and presenting to said physician a possible diagnosis by said base unit.

33. The method as in claim 27, further comprising the step of transmitting and receiving patient data and medical related data over a network connection.

34. An electronic stethoscope system, comprising:
an auscultation converter dimensioned for coupling to a tubing connector of a conventional auscultation head;
a base unit in communication with said auscultation converter;
a sound reproduction element disposed in said base unit, said sound reproduction device configured for reproducing sounds received from said auscultation unit into a form perceptible to a treating physician;
user controls for controlling at least properties of said auscultation converter and said sound reproduction element.

35. The system as in claim 34, wherein said auscultation converter includes a transmitter/receiver element for transmitting bodily sounds picked up by said auscultation head to said base unit, said transmitter/receiver element being wireless.

36. The system as in claim 34, wherein said user controls are implemented as a voice control system for accepting voice commands from a physician and performing functions designated by said voice commands.

37. The system as in claim 34, wherein said sound reproduction element is selected from a group consisting of a video display, loudspeaker, and a combination of both.

38. The system as in claim 37, wherein said video display includes a touch-sensitive screen in communication with said user controls for accepting user commands by way of user selection of displayed options.

39. The system as in claim 34, wherein said system is integrated into a hospital bed.
